(19) United States
(12) Patent Application Publication
(10) Pub. No.: US 2004/0127816 A1
(43) Pub. Date: Jul. 1, 2004

(54) Handheld blood collection device

(76) Inventor: Adriano Braun Galvao, Chicago, IL (US)

Correspondence Address: Pauley Petersen Kinne & Erickson, Suite 365, 2800 W. Higgins Road, Hoffman Estates, IL, 60195, US

(21) Appl. No.: 10/331,937

(22) Filed: Dec. 30, 2002

(51) Int. Cl. A61B 5/00

(52) U.S. Cl. 600/575

(57) ABSTRACT

A handheld device for drawing fluid specimens, such as blood, comprising a structural body forming a manifold channel and a plurality of vial bodies integrated with the structural body, each of the vial bodies forming a port. A connection needle is seated within each of the ports. A valve element is slidably positionable with respect to the manifold channel, and slidably movable between a plurality of positions, each of the positions forming selective fluidic communication between the manifold channel and one port of the vial bodies, and preventing fluidic communication between the manifold channel and each of a plurality of remaining ports of the vial bodies. In one embodiment of this invention, the valve element is slidably movable with respect to the manifold channel to align one of a plurality of apertures formed in the valve element with respect to the manifold channel to form fluidic communication between the manifold channel and one of the ports of the vial bodies and preserve a vacuum formed in a vial connected at each of the remaining vial bodies. A needle is removably connectable to the structural body for drawing blood, for example, from a patient.

### BACKGROUND OF THE INVENTION

1. Field of the Invention

This invention relates to a device for drawing fluid specimens. More particularly, this invention relates to a handheld blood collection device suitable for use by a phlebotomist to selectively obtain at least one blood sample.

2. Discussion of Related Art

Phlebotomy is the practice of opening a vein by puncture to remove blood specimens. The procedure is vital to a patient's health assessment. Laboratory analysis or testing can be used for diagnostic testing and monitoring a patient's health status, for example. The procedure for obtaining blood specimens or samples is a delicate procedure with considerable room for error. It has been estimated that the cost of specimen collection errors in an average 400-bed hospital can be as much as $200,000 per year. Specimen collection errors lead to incorrect test results due to contaminated specimens, patient discomfort and in some cases, death. In a blood room setting, the procedure is stressful; phlebotomists are under pressure to perform many venipunctures and the patient is generally anxious about his or her exam.

During the phlebotomy procedure, a phlebotomist typically complies with an “order of draw,” wherein evacuated vials are filled in a defined sequence to prevent cross-contamination between non-additive vials and additive (anti-coagulant) vials. For example, the order in which multiple vials are collected or how a syringe is used after a draw can affect the results of the test. With the exception of blood cultures or other tests that require special techniques to minimize the possibility of microbial contamination, the order in which the evacuated vials are used is different from the order in which the vials are filled when using a syringe to draw the blood. During the phlebotomy procedure, a phlebotomist may collect a desired volume of blood within a first vial, typically indicated by a color marker, such as a red vial cap, wherein any undesired particles, for example small fragments of the patient's skin or debris on the skin surface, are collected to prevent influence of clotting agents and/or any further contamination of the collected specimens during the procedure.

Many conventional blood collection devices require the removal of a filled vial and the insertion or connection of an empty vial during the blood drawing procedure. This order is designed to reduce interference in specimen testing caused by inadvertently mixing additives between vials. This can occur when blood in a vial that contains an additive makes contact with the needle that punctures the rubber vial top. Blood remaining within the needle may be transferred to the next vial, contaminating that vial, and thus affecting test results on that specimen. Further, connecting the replacement vial while simultaneously supporting the needle inserted into the patient often compromises the patient's comfort.

Additionally, many conventional blood collection devices include vials having determined or calculated vacuum measured to perform a single draw from the blood stream. However, the vacuum formed within the vial may be sufficiently strong to close the vein wall preventing blood from being drawn and collected. For example, many patients may have thin and/or weak veins that collapse during the procedure, resulting in further complications, such as pain and bruising, particularly at the point of insertion of the needle through the patient's skin surface. Further, if more than one evacuated vial is used during the procedure, the vacuum formed within the device may be multiplied by the number of interconnected and non-isolated vials, thus increasing the potential for vein collapse. Additionally, when the multiple vials are interconnected, blood is able to flow between the vials through a main channel. As a result, blood may reflux from the full vials into empty interconnected vials, contaminating the vials with undesirable reagents.

There is an apparent need for a blood collection device that prevents or reduces errors, increases operational efficiency and improves patient care quality.

There is also an apparent need for a handheld blood collection device that can be used to collect a desired number of blood specimens without cross-contamination.

There is also an apparent need for a handheld blood collection device that can be used to collect a desired number of blood specimens while eliminating or reducing the risk of vein collapse.

There is also an apparent need for a handheld blood collection device that requires minimal movement after the blood has begun flowing, to reduce patient discomfort.

### SUMMARY OF THE INVENTION

It is one object of this invention to provide an improved handheld blood collection device.

It is another object of this invention to provide a handheld blood collection device forming selective fluidic communication with one of a plurality of vials mounted to a structural body of the device while preventing cross-contamination or cross-filling of the remaining vials.

It is another object of this invention to provide a handheld blood collection device forming selective fluidic communication with one of a plurality of vials, while maintaining a vacuum formed within each isolated vial mounted to a structural body, thereby preventing collapsing of the patient's vein during a blood collection procedure.

It is another object of this invention to overcome one or more of the problems described above.

The above and other objects of this invention are accomplished with a device for drawing fluid specimens, for example a handheld blood collection device, including a structural body forming a manifold channel. Preferably, the structural body is transparent to allow a phlebotomist administering the procedure or test to observe and control the fluidic communication within the blood collection device during the blood drawing or collection procedure. A phlebotomy needle is removably connectable with respect to the structural body, and forms a passage in fluidic communication with the manifold channel. For example, in one preferred embodiment of this invention, the phlebotomy needle is threadedly connectable to the structural body. Other suitable mechanical connections, such as press-fitting, may be used to removably connect the phlebotomy needle to the structural body.

A plurality of vial bodies are mounted to or integrated with the structural body. Each vial body forms a port in selective fluidic communication with the manifold channel. A plurality of vials are mountable with respect to the structural body for collecting and containing blood drawn from the patient. For example, each vial may be removably positionable within a corresponding port formed by the vial body. A connecting needle is seatable within each port and at least a portion of the connecting needle extends into the corresponding vial removably positioned within the port. Preferably, at least a portion of the connecting needle is covered by a needle cover, for example made of a suitable biocompatable rubberized material, to maintain the device in sterile condition until use.

Further, upon filling a vial with a desired amount of blood, a phlebotomist may remove the filled vial from the port, and the needle cover may move or retract to an initial position thereby covering the connecting needle to prevent blood from exiting the device through an exposed connecting needle and/or undesired contact with the connecting needle.

A valve element is slidably positionable with respect to the manifold channel to direct blood flow into one vial while isolating the remaining vials, thereby preventing cross-contamination between the vials. For example, the valve element may be slidably positioned within a channel or track formed by the structural body and positioned with respect to the manifold channel. The valve element is slidably movable between a plurality of positions, each position forming selective fluidic communication between the manifold channel and one port formed by one vial body. With the valve element in position to selectively form fluidic communication between the manifold channel and one port, fluidic communication between the manifold channel and each of the remaining ports formed by the vial bodies is obstructed. In one preferred embodiment of this invention, a plurality of apertures are formed through the valve element, each of the apertures corresponding to one position. The valve element is slidably movable with respect to the manifold channel to align one aperture with respect to the manifold channel to form fluidic communication between the manifold channel and one port, while preserving a vacuum formed within each vial mounted within each remaining port. Preferably, the valve element includes a plurality of projections and/or indentations corresponding to each position, to register one aperture formed through the valve element with a corresponding port to provide fluidic communication between the manifold channel and the selected port.

### BRIEF DESCRIPTION OF THE DRAWINGS

The features of this invention can be better understood when this specification is read in view of the drawings, wherein:

FIG. 1 is a perspective view of a handheld blood collection device, according to one preferred embodiment of this invention;

FIG. 2 is a perspective view of a portion of the handheld blood collection device of FIG. 1, showing selective fluidic communication between a manifold channel and a port formed in a vial body, according to one preferred embodiment of this invention;

FIG. 3 is a perspective view of a portion of the handheld blood collection device of FIG. 1, showing a vial body integrated with a structural body of the handheld blood collection device and forming a port, according to one preferred embodiment of this invention;

FIG. 4 is a perspective view of a portion of the handheld blood collection device of FIG. 1, showing a needle cover mountable over a connecting needle seated within a port, according to one preferred embodiment of this invention;

FIG. 5 is a perspective view of the valve element positionable with respect to a manifold channel formed in the handheld blood collection device of FIG. 1, according to one preferred embodiment of this invention;

FIG. 6 is a view of the handheld blood collection device being used to draw blood from a patient, according to one preferred embodiment of this invention; and

FIGS. 7-10 illustrate a sequential blood collection procedure, wherein a valve element is positionable with respect to a manifold channel to selectively provide fluidic communication between the manifold channel and one vial chamber, according to one preferred embodiment of this invention.

### DETAILED DESCRIPTION OF PREFERRED EMBODIMENTS

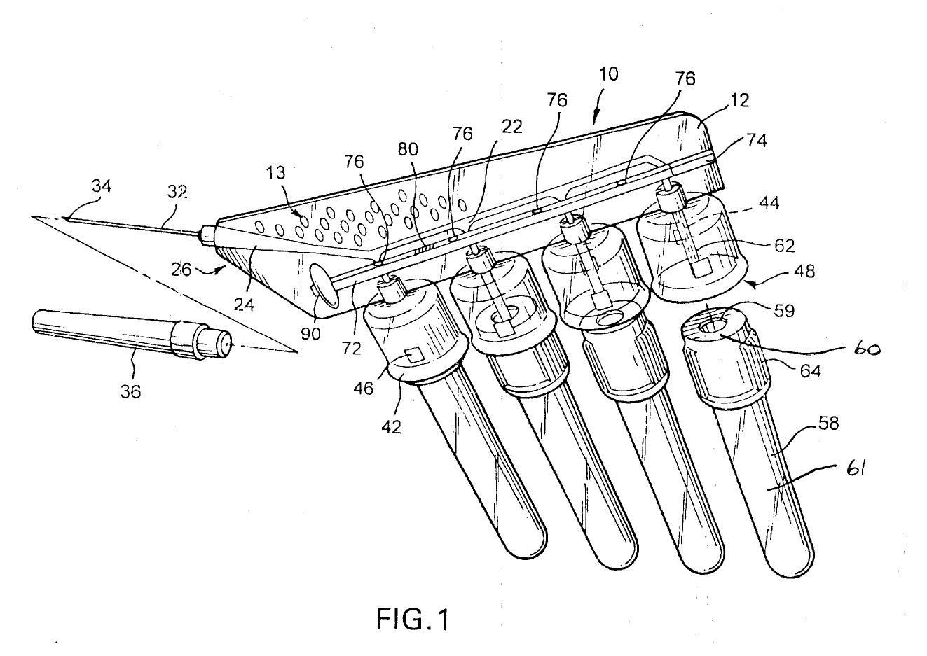

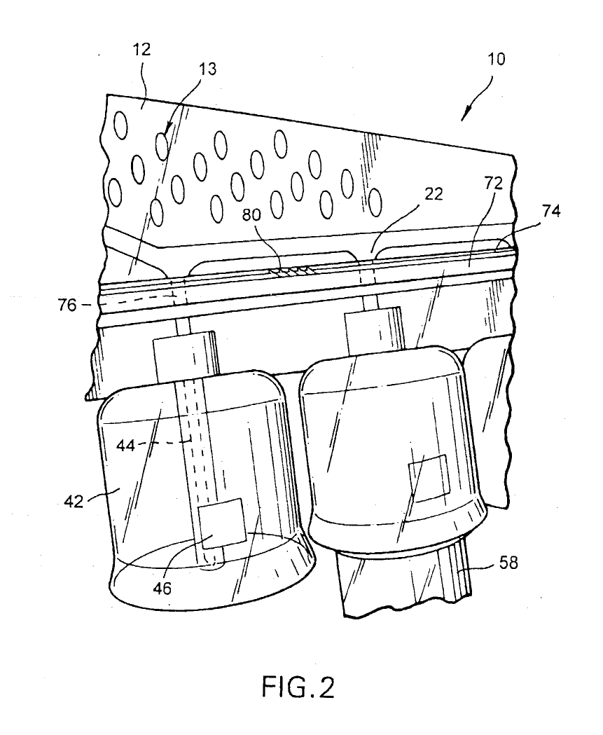

Referring to FIGS. 1-4, the present invention relates to a handheld device for drawing fluid specimens, for example a venesection device or a blood collection device. In one preferred embodiment of the invention, the handheld blood collection device is disposable. Thus, after a collection of a blood samples is obtained, the device may be properly discarded. For purposes of describing the present invention, the device will be described as a handheld blood collection device, for example usable by a phlebotomist to draw blood from a patient for clinical testing. It is apparent to those skilled in the art that the teachings of the present invention may be used for collecting or drawing other bodily fluids during surgical procedures or clinical procedures, for example.

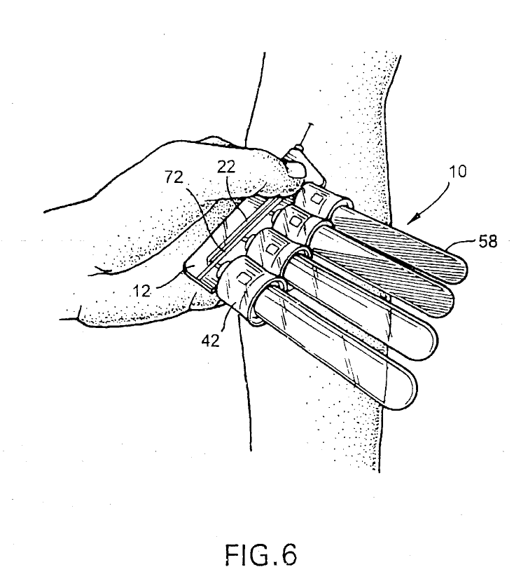

In one preferred embodiment of this invention, the handheld blood collection device 10 comprises a structural body 12. Preferably, but not necessarily, the structural body 12 is transparent in order for the phlebotomist to control the fluidic communication within the handheld blood collection device 10 to properly draw a plurality of blood samples from the patient. As shown in FIG. 1, for example, at least a portion of structural body 12 forms a textured grip surface 13 comprising a plurality of projections and/or indentations that provide frictional interference, for example, to prevent slippage of the phlebotomist's hand as pressure is applied against a patient's skin surface to insert a needle 32 of handheld blood collection device 10 into a patient's vein (FIG. 6). Structural body 12 forms a manifold channel 22, which extends along at least a portion of a length of structural body 12.

In one preferred embodiment of this invention, a needle 32, for example a phlebotomy needle, is removably mountable or connectable with respect to structural body 12, and forms a passage 34 in fluidic communication with manifold channel 22. For example, needle 32 may be threadedly connectable to structural body 12, as shown in FIG. 1. Other suitable mechanical connections, such as press-fitting, may be used to removably connect needle 32 to structural body 12 to form or provide fluidic communication between passage 34 and manifold channel 22. As shown in FIG. 1, needle 32 is mountable or positionable within a funneled channel 24 formed by at least a portion of manifold channel 22 at a first end portion 26 of structural body 12 to form or provide fluidic communication between passage 34 formed within needle 32 and manifold channel 22. Needle 32 may comprise any suitable needle for performing the desired action, such as drawing blood from a patient or collecting fluid specimens. For example, needle 32 may comprise a hypodermic needle or a butterfly needle. Preferably, but not necessarily, a needle cap 36 is positionable over needle 32 to prevent undesired contact with needle 32, such as undesired sticking or pricking of a patient's or phlebotomist's skin surface with needle 32, when handheld blood collection device 10 is not being used to draw blood for example.

Referring to FIGS. 1-4, in one preferred embodiment of this invention, a plurality of vial bodies 42 each is mounted with respect to structural body 12, and forms a port 48 in selective fluidic communication with manifold channel 22. Preferably, each vial body 42 is integrated with structural body 12. It is apparent to those skilled in the art that vial body 42 may be connected or mounted with respect to structural body 12 using any suitable means, mechanical or otherwise. Each port 48 has suitable dimensions to accept and securely position a vial 58 removably connectable or mountable to corresponding vial body 42. In one embodiment of this invention, at least one vial body 42 may form a port 48 having different dimensions than the remaining ports 48, such as a different inner diameter or different depth, for accepting a vial 58 having different dimensions from the remaining vials 58 mounted within ports 48, such as a different outer diameter. Each port 48 may also accommodate more than one vial 58 using suitable mechanical connections, for example.

Each vial 58 is securely connected to a corresponding vial body 42, for example securely positioned within a corresponding port 48. A vacuum within vial 58 is maintained unless or until vial 58 is selected by the phlebotomist to be filled with drawn blood. As set forth above, maintaining the vacuum formed within each vial 58 may be important to prevent damage to the patient's veins during the blood drawing procedure, such as collapsing of the selected vein and/or systemic veins, as seen in prior art blood collection devices. Additionally, maintaining the vacuum formed in each unselected vial prevents contamination of the blood samples by simultaneously cross-filling more than one vial 58.

For example as shown in FIG. 1, in one preferred embodiment of this invention, each vial 58 forms an opening 59 and a chamber 61 for containing or holding blood or fluid specimens drawn or collected from a patient by the phlebotomist. Further, vial 58 may form a sleeve or gasket 60 covering at least a portion of opening 59 formed by vial 58, to prevent contaminants or particles from entering vial 58 and contaminating the drawn and collected blood samples. Sleeve 60 also prevents any drawn and collected blood from undesirably exiting or spilling from opening 59.

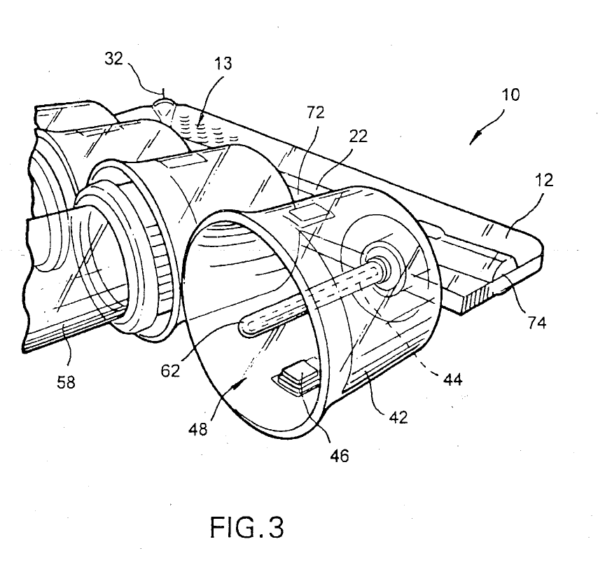

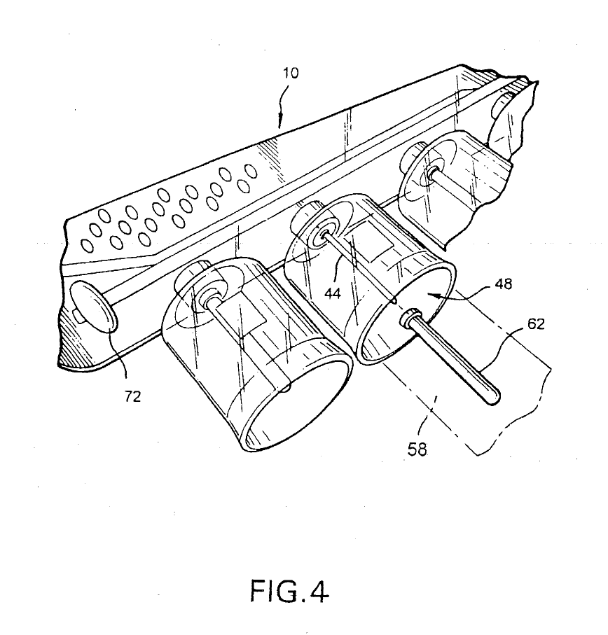

As shown in FIG. 1, each vial body 42 may include a connecting needle 44 positioned within vial body 42. Preferably, connecting needle 44 is seatable within port 48. Referring further to FIGS. 3 and 4, a needle cover 62 is mounted or positioned over connecting needle 44 to maintain connecting needle 44 in a sterile condition and prevent contamination of handheld blood collection device 10, as well as prevent undesired contact with connecting needle 44. Needle cover 62 may comprise any suitable material, for example a biocompatable rubber material. As vial 58 is positioned within port 48 and mounted to vial body 42, sleeve 60 frictionally contacts needle cover 62 to expose a passage formed through connecting needle 44 and form selective fluidic communication between chamber 61 formed by vial 58 and manifold channel 22. Preferably, at least a portion of connecting needle 44 extends into vial 58. Vial 58 is removably connected to vial body 42 so that vial 58 can be easily removed from within port 48 after collecting drawn blood. Upon collection of a desired amount of blood, vial 58 is removed from within port 48 and needle cover 62 is movable or retractable to an initial position, wherein needle cover 62 is positioned over and/or covers connecting needle 44 to prevent undesired exiting of blood from connecting needle 44 and undesired contact with connecting needle 44 and blood on or contained within connecting needle 44.

In one preferred embodiment of this invention, as shown in FIGS. 1-3, in order to securely mount vial 58 within port 48 of vial body 42, vial body 42 forms at least one projection 46 that interferes with vial 58 as vial 58 is positioned within port 48. For example, as shown in FIG. 3, projection 46 may extend into port 48. Preferably, projection 46 is biased to maintain secure contact or engagement with vial 58. Additionally or alternatively, vial 58 may form at least one projection or group of projections and/or indentations 64 as shown in FIG. 1, which interfere with at least a portion of an inner surface of vial body 42 forming port 48. Preferably, vial 58 is securely and tightly positioned within port 48 to preserve a vacuum created within vial 58, as discussed above. It is apparent to those skilled in the art that vial body 42 may form one or more projections 46 and/or indentations, each interfering with a corresponding portion of vial 58 to securely position vial 58 within port 48. Similarly, vial 58 may form one or more projections and/or indentations 64, each interfering with a corresponding portion of vial body 42 to securely position vial 58 within port 48.

As shown in FIGS. 1-5, in one preferred embodiment of this invention, handheld blood collection device 10 further comprises a valve element 72 slidably positionable with respect to manifold channel 22 to selectively form fluidic communication between manifold channel 22 and one port 48 wherein connecting needle 44 is positioned, while maintaining or preserving a vacuum formed within each vial 58 connected or mounted within the remaining ports 48. For example, valve element 72 may be slidably positioned within a channel or track 74 formed by structural body 12. Because valve element 72 selectively forms fluidic communication between manifold channel 22 and one port 48, the occurrence of multiplying the vacuum within manifold channel 22 is prevented. The multiplying vacuum condition is one problem associated with conventional blood collection devices, and may cause a patient's vein to collapse, preventing proper drawing of the patient's blood and perhaps undesirable health risks or complications to the patient.

Referring to FIGS. 1, 2 and 5, in one preferred embodiment of this invention, valve element 72 forms a plurality of apertures 76 through valve element 72. Valve element 72 is slidably movable within track 74 to align or register at least a portion of only one of the plurality of apertures 76 with respect to manifold channel 22 to selectively form fluidic communication between manifold channel 22 and only one port 48 of the plurality of formed ports 48, corresponding to or aligned with aperture 76. In one preferred embodiment of this invention, valve element 72 is movable to control fluidic communication formed between manifold channel 22 and the one port 48. For example, a portion of aperture 76 may be aligned with respect to manifold channel 22 to form partial fluidic communication between manifold channel 22 and the one port 48. With the aperture 76 at least partially aligned with one port 48, the remaining apertures 76 formed by valve element 72 do not align with a corresponding port 48 of the remaining ports 48, thus preventing fluidic communication between the manifold channel 22 and the remaining ports 48. As a result, a vacuum formed within the corresponding vials 58 positioned within the remaining ports 48 is maintained or preserved.

In one preferred embodiment of this invention, valve element 72 is slidably movable between a plurality of dedicated positions, each of the dedicated positions forming selective fluidic communication between manifold channel 22 and one port 48, and preventing fluidic communication between manifold channel 22 and each of the plurality of remaining ports 48 formed by corresponding vial bodies 42. Each aperture 76 corresponds to one of the dedicated positions. Preferably, valve element 72 comprises at least one projection and/or at least one indentation 80 that interferes with a portion of track 74 and/or manifold channel 22 at a dedicated position to selectively position valve element 72 with respect to manifold channel 22. For example, one aperture 76 is aligned with one corresponding port 48 to selectively form fluidic communication between manifold channel 22 and one port 48. Preferably, valve element 72 comprises a plurality of sequential projections and/or indentations 80. Each sequential projection and/or indentation 80 corresponds with one aperture 76 formed through valve element 72.

In one preferred embodiment of this invention, valve element 72 allows the phlebotomist to precisely control the vacuum within each vial 58. For example, if a patient's vein is too small, and the possibility of vein collapse is present, the phlebotomist can control the opening or fluidic communication provided by the aperture 76 by sliding the valve element 72 within track 74, thereby controlling vacuum intensity. In contrast to the handheld blood collection device 10 of the present invention, when typical conventional devices are used to draw and collect blood samples, if a vein from which the blood is being drawn collapses, the phlebotomist must remove the device and select another vein from which to draw blood, resulting in further patient discomfort.

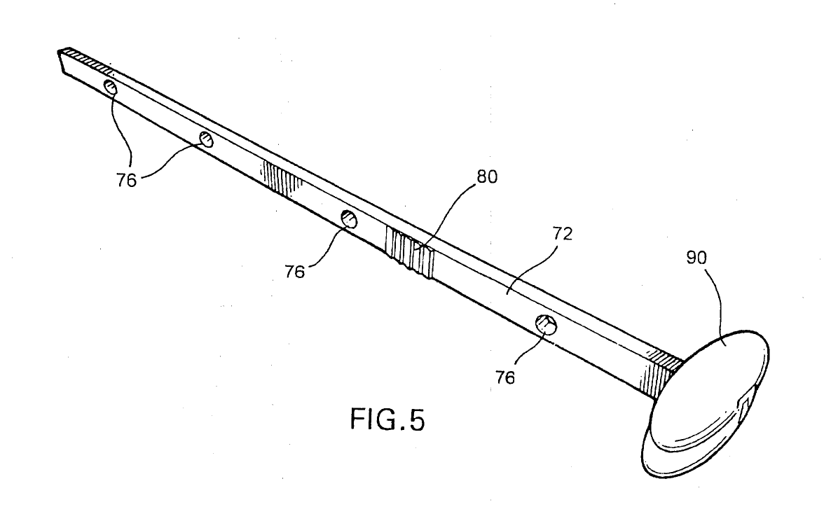

In one preferred embodiment of this invention, a locking mechanism 90 as shown in FIG. 5, for example a depressable biased button, is formed or mounted with respect to structural body 12 and valve element 72 for locking valve element 72 in a dedicated position to prevent movement of valve element 72 within track 74 while blood is drawn through a selected port 48. Locking mechanism 90 may be depressed, for example by the phlebotomist applying pressure to locking mechanism 90 with a finger, to allow selective movement of valve element 72 within track 74 with respect to manifold channel 22. Preferably, locking mechanism 90 is depressable from either opposing surface of structural body 12, depending upon whether the phlebotomist is left-handed or right-handed for example.

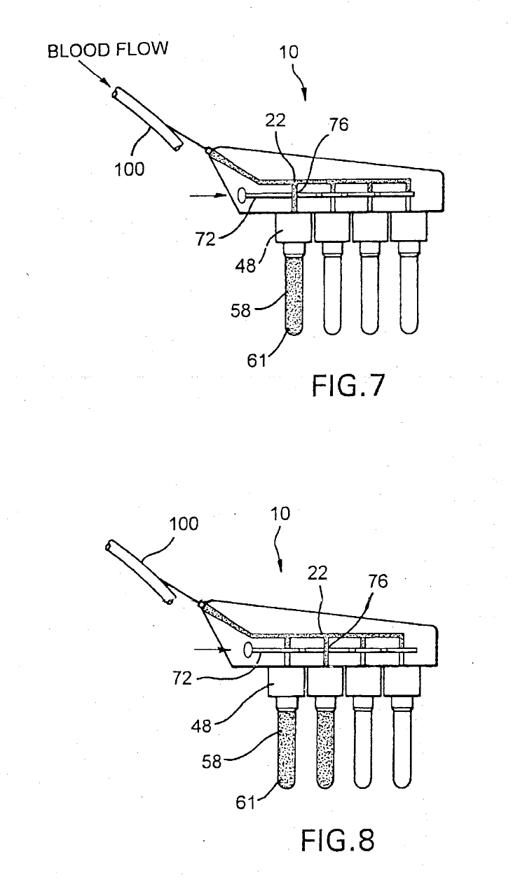

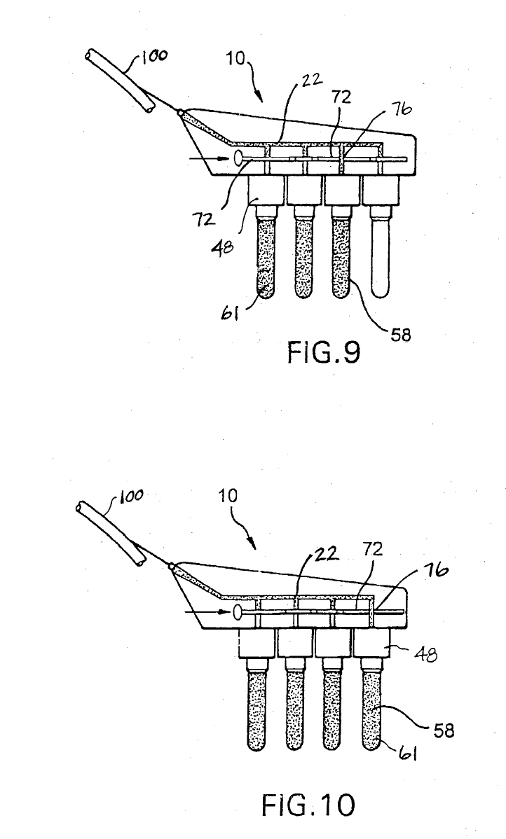

Referring to FIGS. 6-10, the phlebotomist performs the blood collecting procedure using handheld blood collection device 10 as illustrated. With valve element 72 in a first dedicated position as shown in FIG. 7, a first aperture 76 is aligned with manifold channel 22 and a first port 48 to form fluidic communication between manifold channel 22 and a first chamber 61 formed by a first vial 58. With needle 32 inserted into a patient's vein 100, the patient's blood is drawn from vein 100 through manifold channel 22 and the first port 48. The blood is collected within the first chamber 61 of the first vial 58. After the first vial 58 collects a desired volume of drawn blood, valve element 72 is sequentially slidably moved to a second dedicated positioned as shown in FIG. 8, indicated or registered by a corresponding projection and/or indentation 80, wherein a second aperture 76 formed by valve element 72 is aligned with manifold channel 22 and a second port 48 to form selective fluidic communication between manifold channel 22 and a chamber 61 formed by a second vial 58. The patient's blood is drawn through the second port 48 and collected within the second vial 58. Additional vials 58 may be filled with a desired volume of drawn blood as required for clinical testing and analysis. For example, with valve element 72 positioned in a third dedicated position (FIG. 9), a desired volume of blood may be collected within a third vial 58. Similarly, with valve element 72 positioned in a fourth dedicated position (FIG. 10), a desired volume of blood may be collected within a fourth vial 58.

The elements of this invention can be constructed of any suitable biocompatable material, including but not limited to biologically safe plastic and/or rubber materials, metal materials, non-metal materials, or any suitable composite material. Different suitable materials for construction of handheld blood collection device 10 are known to those skilled in the art of medical device design, construction and manufacture.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

What is claimed is:

1. A handheld blood collection device comprising:
a structural body forming a manifold channel;
a needle removably mounted with respect to said structural body, said needle forming a passage in fluidic communication with said manifold channel;
a plurality of vial bodies mounted with respect to said structural body, each of said vial bodies forming a port in selective fluidic communication with said manifold channel; and
a valve element slidably positionable with respect to said manifold channel to selectively form fluidic communication between said manifold channel and one port.

2. The handheld blood collection device of claim 1 further comprising a plurality of vials, each of said vials removably connectable to one of said vial bodies.

3. The handheld blood collection device of claim 1 further comprising a connecting needle seatable within said port.

4. The handheld blood collection device of claim 3 wherein at least a portion of said connecting needle extends into a chamber formed by a vial removably connected to said vial body.

5. The handheld blood collection device of claim 3 further comprising a rubberized needle cover positionable over at least a portion of said connecting needle.

6. The handheld blood collection device of claim 1 wherein said valve element forms a plurality of apertures through said valve element, and said valve element is slidably movable to align one of said apertures with respect to said manifold channel to selectively form fluidic communication between said manifold channel and one port of said vial bodies.

7. The handheld blood collection device of claim 6 wherein said valve element further comprises a plurality of sequential indentations, and each of said sequential indentations corresponds with one of said apertures.

8. The handheld blood collection device of claim 1 wherein said valve element is movable to control said fluidic communication between said manifold channel and one port.

9. The handheld blood collection device of claim 1 wherein at least a portion of said structural body is transparent.

10. The handheld blood collection device of claim 1 wherein said needle is threadedly connectable to said structural body.

11. The handheld blood collection device of claim 1 wherein said needle is press-fitted to said structural body.

12. The handheld blood collection device of claim 1 wherein said port further comprise at least one projection interfering with a vial positionable within said port.

13. The handheld blood collection device of claim 12 wherein said at least one projection is biased to securely engage said vial within said port.

14. The handheld blood collection device of claim 1 wherein said valve element further comprises at least one projection, said at least one projection selectively positioning said valve element with respect to said manifold channel to selectively form fluidic communication between said manifold channel and one port and maintain a vacuum formed within a chamber formed by a vial positioned within each of said remaining ports.

15. The handheld blood collection device of claim 1 wherein at least a portion of said structural body forms a textured surface.

16. The handheld blood collection device of claim 1 wherein said needle comprises one of a butterfly needle and a hypodermic needle.

17. The handheld blood collection device of claim 1 wherein one vial body accommodates a vial of a plurality of vials having a different outer diameter than an outer diameter of each of remaining said vials.

18. A device for drawing fluid specimens comprising:
a structural body forming a manifold channel;
a plurality of vial bodies integrated with said structural body, each of said vial bodies forming a port; and
a valve element slidably positionable with respect to said manifold channel, said valve element slidably movable between a plurality of positions, each of said positions forming selective fluidic communication between said manifold channel and one port of said vial bodies, and preventing fluidic communication between said manifold channel and each of a plurality of remaining ports of said vial bodies.

19. The device of claim 18 further comprising a plurality of apertures formed through said valve element, each of said apertures corresponding to one of said positions.

20. The device of claim 19 wherein said valve element is slidably movable with respect to said manifold channel to align one of said apertures with respect to said manifold channel to form fluidic communication between said manifold channel and one of said ports of said vial bodies and preserve a vacuum formed within a chamber formed by a vial positioned within each of remaining said ports.

21. The device of claim 18 wherein said valve element further comprises a plurality of indentations, each of said indentations corresponding to one of said positions.

22. The device of claim 18 further comprising a phlebotomy needle removably connectable with respect to said structural body, and forming a passage in fluidic communication with said manifold channel.

23. The device of claim 22 wherein said phlebotomy needle is threadedly connectable to said structural body.

24. The device of claim 22 wherein a portion of said manifold channel forms a funneled channel, said phlebotomy needle positionable within said funneled channel.

25. A venesection device comprising:
a structural body forming a manifold channel;
a phlebotomy needle threadedly connectable with respect to said structural body, said phlebotomy needle forming a passage in fluidic communication with said manifold channel;
a plurality of vial bodies mounted with respect to said structural body, each of said vial bodies forming a port; and
a valve element slidably positionable with respect to said manifold channel, said valve element forming a plurality of apertures,
wherein said valve element is slidably movable to selectively align one of said apertures with respect to said manifold channel to selectively form fluidic communication between said manifold channel and one port formed in one of said vial bodies.

26. The venesection device of claim 25 wherein said valve element is slidably movable between a plurality of positions, each of said positions corresponding to one of said apertures to selectively form fluidic communication between said manifold channel and said one port formed in one of said vial bodies.

27. The venesection device of claim 25 further comprising a plurality of vials, each of said vials removably connectable to one of said vial bodies.
